(19) United States
(12) Patent Application Publication
(10) Pub. No.: US 2002/0019588 A1
(43) Pub. Date: Feb. 14, 2002

(54) Frontal electrode array for patient EEG signal acquisition

(76) Inventors: Dominic P. Marro, North Andover, MA (US); Julie E. Boylston, Arlington, MA (US); Thomas G. Clark, Nashua, NH (US); Dawn E. Frazer, Pepperell, MA (US); James W. Nicholson, Dracut, MA (US)

Correspondence Address: Williams & Associates, Suite 300, 1030 Fifteenth Street, N.W., Washington, DC, 20005-1501, US

(21) Appl. No.: 09/887,025

(22) Filed: Jun. 25, 2001

Related U.S. Application Data
(60) Provisional application No. 60/213,642, filed on Jun. 23, 2000.

(51) Int. Cl. A61B 5/04

(52) U.S. Cl. 600/383; 600/393; 600/544

(57) ABSTRACT

A medical appliance for acquiring EEG signals from a patient's head applied solely to the patient's forehead comprising a frontal array of hydrogel electrodes. The appliance also comprises a clear dielectric layer, an electromagnetic shield layer, and signal traces from the electrodes to an appliance connector. The electrodes are generally rectangular in shape and are located at frontal positions according to the International 10-20 System. The electrodes further comprise a retaining mechanism and internal volcano tips to abrade the outer layers of the dermis for improved electrical contact and lower contact impedance.

### FIELD OF THE INVENTION

The current invention relates to the field of medical anesthesia. More particularly, it relates to the field of electronic monitoring of patients undergoing anesthesia, especially during and after surgical operations. The invention relates specifically to the use of electroencephalograph (EEG) signals for electronically monitoring a patient's state of awareness. Most particularly, it relates to removable electrode appliances which acquire EEG signals from the skin of the patient's head.

### BACKGROUND OF THE INVENTION

In current medical practice, at least for lengthy invasive surgery, a patient is placed under general anesthesia. Anesthesiology is a medical art practiced in industrialized countries typically by board certified anesthesiologist-physicians and sometimes by nurse anesthetists. These anesthesia professionals are specifically trained to administer anesthetic drugs and to monitor patients under anesthesia.

The state of patient anesthesia sufficient for surgery is attained by the controlled administration of various drugs with known anesthetic properties. These include one or more vapors or gases which are inhaled or soluble anesthetic drugs introduced intravenously. Volatile substances include nitrous oxide, sevoflurane, desflurane, flurane and isoflurane, and halothane. Intravenous anesthetics include pentothal, evipal, procaine, nitrous narcotic with propofol induction, methohexital, and etomidate.

These drugs are intended to cause the patient to lose consciousness, sensation, and motor control. A correctly administered general anesthetic should remove any sensation of pain and any awareness of the operation itself. The anesthetic should further disable the patient's motor control so that the patient cannot move. Otherwise, the patient may exhibit involuntary (reflex) muscle movements, which can disturb the area being surgically manipulated.

Prevention of movement can be accomplished by anesthetic agents acting on the central nervous system or by means of a blockade of the neuromuscular junction with muscle relaxants. Finally, the anesthesia administration must avoid depressing the patient's blood pressure so much as to reduce blood flow to the brain to a dangerous extent. Generally 50 mm Hg for mean arterial pressure is a lower limit.

Normally an anesthesia professional will monitor the patient's state of awareness by means of a number of disparate clinical signs known empirically to provide useful and reliable information about the patient's state of unconsciousness. The anesthesia professional will monitor the patient's vital signs, such as respiration and pulse rates, check the patient's pupil dilation, and check certain reflexes, such as the lash reflex, and other physiological signs, and from these qualitative features and based on experience estimate the depth of anesthesia.

In some instances, however, either the practitioner does not have access to all of the required clinical information or other circumstances intervene. For example, in some procedures the patient is draped in such a way as to make observation of some clinical indicators difficult or impossible.

In these and other circumstances it would be advantageous to have an electronic monitor to track the patient's level of consciousness. In particular, an instrument, which, once the plane of anesthesia is established qualitatively by the anesthesiologist using traditional clinical indicators, would indicate significant changes in the patient's state of anesthesia or patient responses to stimuli, which would indicate insufficient anesthesia, would be highly adantageous. Patients who have drifted out of sufficiently deep anesthesia have reported terror at becoming aware of the ongoing surgical procedure while paralyzed.

A number of inventors have developed systems for using EEG signals to monitor anesthesia, sleep, or other states on the consciousness-unconsciousness continuum. In particular, inventors, including one of the inventors herein, developed a system for electronic anesthesia monitoring, “Anesthesia Monitoring System Based On Electroencephalographic Signals,” U.S. patent application Ser. No. 09/431,632, filed Nov. 2, 1999 (incorporated herein by reference as though fully set forth) (also European Patent Application No. 01 109 804.3), which uses solely EEG signals acquired from the skin of the patient's head to produce a displayed index of the patient's state of awareness or anesthesia. Other systems exist which use EEG signals or EEG signals in combination with other bodily parameters from the patient to gauge level of anesthesia.

What these systems have in common is the need to acquire very faint electrical signals representative of the patient's EEG signals from the skin of the patient's head. A common means of acquiring signals representative of electrical activity of bodily functions in humans is via receptors, specifically electrodes, applied to the patient's skin. Monitoring brain activity, in particular, typically requires a plurality of electrically-connected receptors to be applied to predetermined, anatomically-well defined sites on the skin of the patient's head.

In the recent past, large numbers of electrodes were used to record electrical activity of the human brain. Typical monitoring systems employed 21 electrodes mounted on the patient's head according to specific systems, primarily the International 10-20 system. It was common for some time to employ electrodes in sets of 21, as for example in U.S. Pat. No. 5,497,934. For certain applications, however, it is natural to conjecture that such a large number of electrodes might be unnecessary. Indeed, where the purpose of monitoring patient brain activity is to trace the level of consciousness in a patient receiving general anesthetic and thereby control the amount of anesthetic being administered, receptors than are not absolutely necessary for performing the desired patient monitoring function may become a distinct liability.

One of the inventors of the current invention with other inventors developed an appliance for placing a more limited set of electrodes to the patient's head, “Self Adjusting Headgear Appliance Using Reservoir Electrodes,” U.S. Pat. No. 6,128,521, issued Oct. 3, 2000 (incorporated herein by reference as though fully set forth)(also European Patent Application No. 99935484.8). That invention took advantage of the fact that analysis of multiple channels of EEG information showed that much of the information obtained from, e.g., 21 channels was redundant. That invention therefore reduced the number of channels to seven.

A significant disadvantage of this previous invention was that it was still complex and relatively expensive to manufacture. It comprised 7 leads which had to be affixed to different and very specific locations on the patient's head prior to the surgical procedure. Anterior (frontal), central and posterior (mastoid) electrode sites in this array were attached to both hairy and hairless areas of the scalp, thus requiring the use of different electrode types and of tensioning elements to assure reliable contact with the patient's skin.

Designing a device for acquisition of physiological signals for patient monitoring in the operating room (OR) and intensive care unit (ICU) presents other significant design challenges as well. One significant design barrier for such a device is the OR environment. The harsh electromagnetic environment of the OR challenges any signal acquisition system, especially for electroencephalograph (EEG) signals, since electro-surgical devices produce stray induction signals with power up to 1 billion times the power of the EEG signal. In order to minimize the coupling of these undesired signals to EEG sensors and lead wires, shielded lead wires and short sensor leads are standard practice. Superior electronics in the monitoring device, especially at the pre-amplifier stage, can largely suppress electromagnetic induction (EMI). There are situations, however, where electrocautery occurs in close proximity to the patient mounted signal electrodes and signal corruption may occur. Thus there is need for additional stray signal suppression in the electrode appliance itself.

Another significant design problem is to assure that the appliance being utilized is matched with the application running in the instrument. One solution to this problem is to encode in the appliance or the cable the identity (model) of the appliance. The appliance model number is then associated with specific application software ensuring intended system operation. Moreover, problems in the field are more easily resolved if inadequate performance can be traced to lot number and date of manufacture. Persistent problems with electrode impedance for example, when associated with date and lot code, will provide a means to monitor and remedy quality and performance related problems. The same means can be used to identify products whose shelf life has expired. Providing the user a means of automating the capture of device specific manufacturing information creates a reliable means for monitoring the quality of disposable appliances.

Finally, there is a need to render patient-applied disposable devices not reusable. It is known that from time to time users of disposable, single use devices reuse the device, thereby exposing the patient to risk of infection. Substandard instrument performance may also be a result.

It is therefore an object of the current invention to provide an appliance comprising electrodes for acquisition of EEG signals from a patient's head using 5 or fewer, and at most 6, electrodes mounted only on the patient's forehead instead of the prior configuration using 7 electrodes deployed both anterior and at posterior locations. It is a further object of this invention to do away with completely the use of tensioning elements necessary for arrays which have posterior contacts. It is a further object to provide a device which would inherently cost less to manufacture by virtue of using less material, avoiding costly secondary manufacturing operations, and simplifying packaging. It is yet a further object to provide a device which is highly resistant to electromagnetic induction of spurious signals from, for example, electrocautery devices. It is also a further object of this invention to provide a device which provides internal identification so that the appliance is properly matched with the monitoring system. It is a further object to provide unit traceability. It is yet a further object to provide a device which is disposable. Finally, it is a further object to provide a device which is non-reusable and/or resists reuse on a second patient.

### SUMMARY OF THE INVENTION

The current invention is an appliance comprising an array of electrodes for acquiring EEG signals solely from a patient's forehead. It comprises at most six electrodes, and preferably fewer, all attached to the patient's forehead. The device additionally comprises a connector enabling it to be attached to a cable which transmits signals to a monitoring system. The electrodes comprise an adhesive portion, conducting hydrogel, and a matrix for containing the hydrogel prior to application to the patient. The electrode further comprises means for assuring that the hydrogel makes electrical contact with the patient's skin. Preferably the electrode also comprises volcano tip reservoirs for conducting hydrogel.

### BRIEF DESCRIPTION OF THE DRAWINGS

FIG. 1 depicts a schematic of the configuration of the system incorporating the frontal electrode array.

FIG. 2 portrays detail of the electrode arrangement on the frontal electrode array.

FIG. 3A shows detail of a typical electrode on the frontal electrode array.

FIG. 3B illustrates the use of sponge trapping to provide superior electrode performance.

FIG. 4 shows the geometry of the frontal array appliance.

FIG. 5 illustrates the configuration of the patient interface/cable connector.

### DESCRIPTION OF THE PREFERRED EMBODIMENT

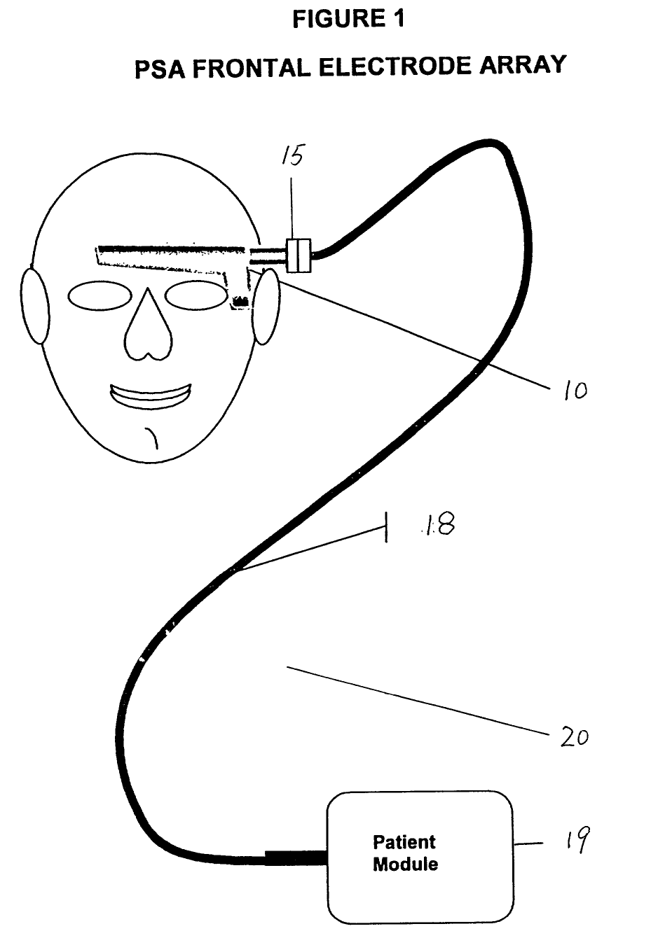

With specific reference to FIG. 1, in the basic configuration of the frontal appliance 10 as used in the patient portion of a monitoring system 20, the appliance 10 is applied to the patient's forehead. It uses a connector or interface 15 to make electrical contact with a patient cable 18. The patient cable 18 is further connected to a Patient Module 19, which in some embodiments performs preliminary analysis of signals and in any event functions to relay the signals to the Monitor (not shown).

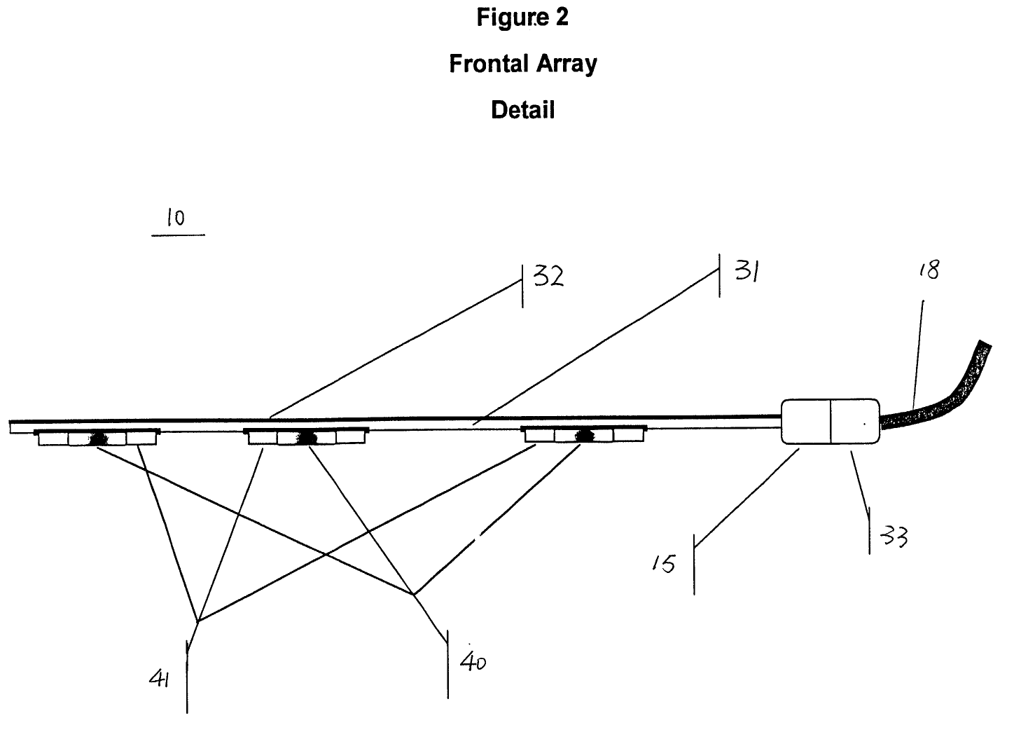

The frontal array appliance 10 with arrangement of electrodes 40 is shown schematically in FIG. 2. With specific reference to FIG. 2, the appliance comprises a non-conductive substrate 31. In order to reduce the coupling of external electromagnetic fields to the frontal array electrodes 30, a conductive shield layer 32 is laminated onto the top surface (away from the patient) of the appliance 10. The shield layer 32 is attached to the cable shield and or ground/drain wire through the appliance connector 15 and the cable connector 33. This shield on the outside in combination with the patient as a signal ground on the inside establishes effectively a Faraday shield around the frontal array signal electrodes 30. This design significantly reduces the interference from electromagnetic fields generated by, e.g., electrocautery devices, and from varying electric and low frequency electric fields from line powered devices. Low frequency fluctuations caused by static charges on OR personnel is also significantly reduced. The shield layer 31 is made of a conductive laminate applied to the top surface of the appliance or by the application of conductive ink comprising metallic silver or silver in combination with silver chloride. This conductive surface 31 connects to the patient cable 18 shield, shield drain wire and or patient ground through mating connectors on the appliance 115 and patient cable 33.

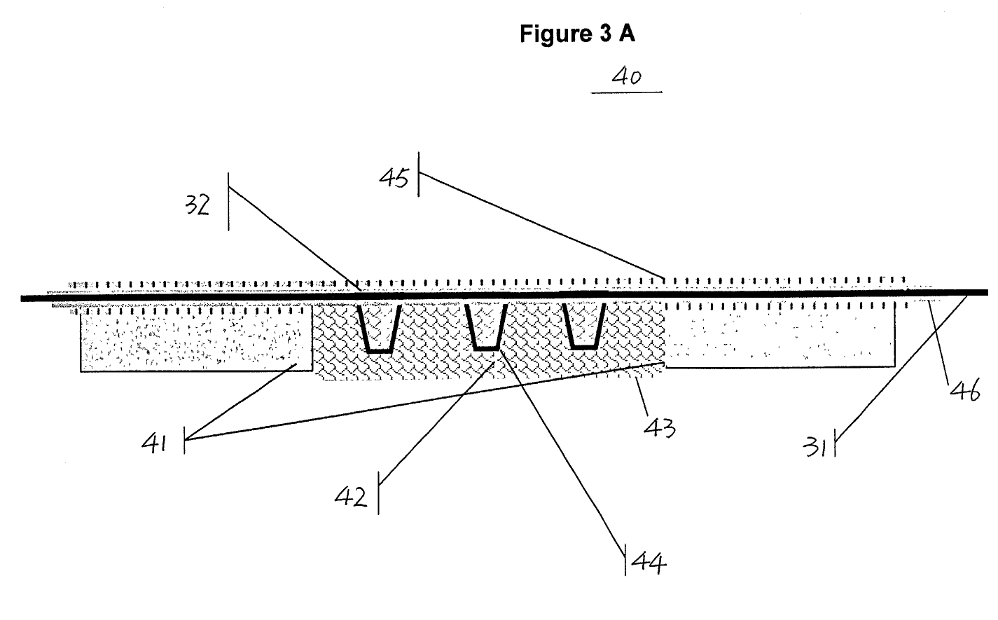

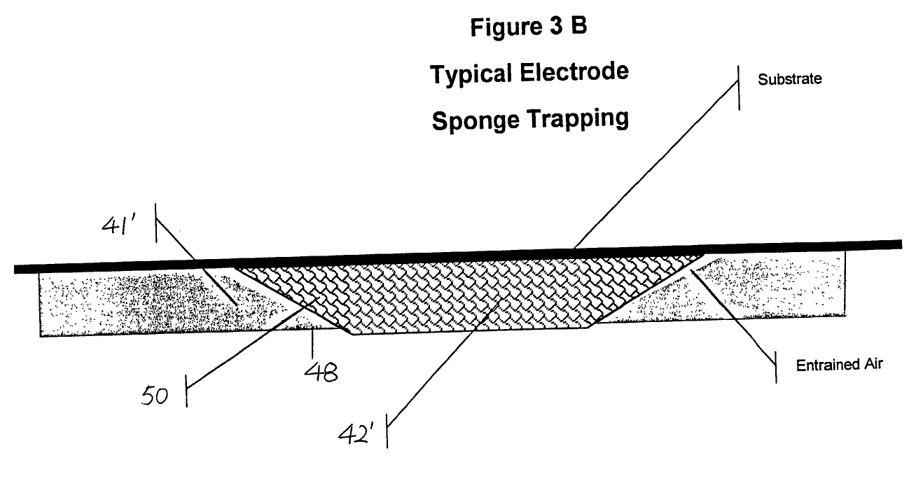

In the most preferred embodiment, as shown in FIG. 3A, a typical electrode 40 comprises an element of resilient foam material 41, adhesive on the side proximal to the patient, generally in the shape of an annulus. Inside the annular adhesive foam 41 is an element of open cell foam 42 impregnated with conductive hydrogel 43. In the most preferred embodiment, this open cell foam element additionally comprises relatively stiff volcano tips 44, the function of which is to abrade or part the dead skin layer of the dermis and cause mild abrasion thereof in order to permit more effective electrical contact between the conducting hydrogel and the underlying live skin. This feature also reduces preparation time significantly. In addition to the non-conducting flexible substrate 31, this element comprises the conductive coating or laminate which serves as a shield 32, an external dielectric layer 45 covering and protecting the shield layer 32, and a conductive signal trace 46, which conducts the electrical signals from the conducting hydrogel electrode to the appliance connector 15.

In the most preferred embodiment, the non-conducting flexible substrate 31 is clear, that is, see-through. This feature allows for monitoring of the patient's skin for potential irritation under the electrode.

In a slightly different embodiment, shown in FIG. 3B, the annular adhesive foam pad 41′ has an inner surface which, in cross section, forms an acute angle with the horizontal 48 on the side proximal to the patient, and the inner element of hydrogel impregnated open cell foam or sponge 42′ forms a less acute or obtuse angle with the horizontal on the side distal to the patient, such that an entrained air space 50 is formed between the adhesive pad and the hydrogel electrode. The entrained air space 50 allows for gel dispersion within the captured sponge. This feature minimizes gel dispersion between the adhesive pad and the skin, thereby maximizing the contact area of the skin with the adhesive pad. This feature promotes secure skin adhesion of the adhesive pad. It further secures the sponge in place so that the sponge will not be inadvertently left on the patient when the device is removed.

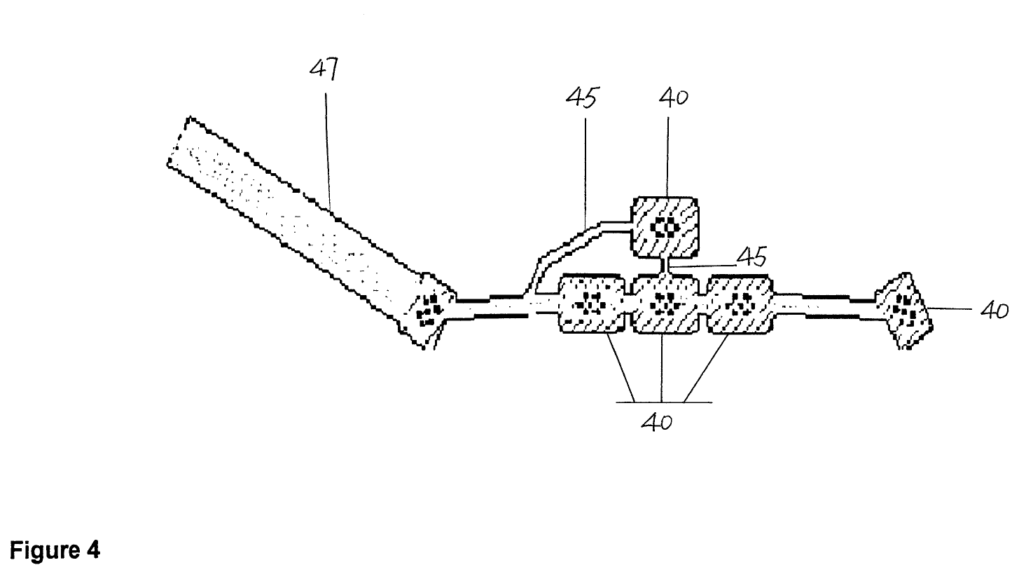

The general geometry of the frontal array appliance is shown in FIG. 4. The electrode 40 preferably has a rectangular shape in contrast to the traditional round shape. This configuration maximizes the hydrogel to skin contact area for a given smaller transverse dimension while permitting electrode placement in accordance with the international 10-20 system. This arrangement also permits the appliance to fit on small adult heads. Thin members 45 connect sites F7 to Fp1 and F8 to Fp2. The thin flexible member accommodates a wider range of head sizes. The flexible member also loops to accommodate smaller heads. Additionally, the long pigtail leading 47 to the cable connector assures that the cable is away from the patient. This feature potentially prevents sores which might be produced if the patient's body were in contact with the cable for extended periods of time. The pigtail 47 may also be folded to minimize the size of the appliance before use.

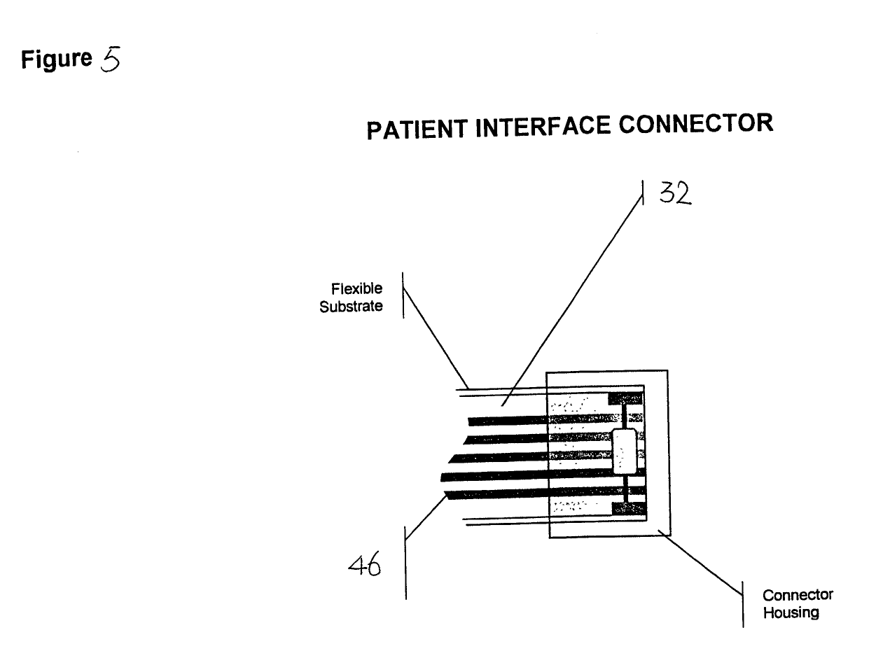

As shown in FIG. 5, the appliance connector terminates the three to five signal traces 46. The shield layer 32 is electrically connected so that when the connector mates with the patient cable 18 the shield layer becomes grounded.

Also in the most preferred embodiment, the frontal array appliance incorporates an externally addressable, single wire “solid state serial number”, such as the DS2401 manufactured by Dallas Semiconductor (not shown). This feature provides the ability to record uniquely the manufacturer, model number, serial number and other information pertinent to the identification of the frontal array appliance unit. Such a solid state serial number, among other things, enables quality monitoring. Individual unit performance can be related to serial number, lot number date code and other information necessary for monitoring product quality. In addition, the device can be uniquely associated with a specific software application in the anesthesia monitoring system. The use of an appliance with a digital serial number enables automatic loading of the appropriate operating room or intensive care application software automatically, minimizing the likelihood of operator error. Further, a date code can be compared to the system clock to prevent the use of a device whose shelf life has expired. Finally, versions of this solid state serial number are available that can be rendered inoperative after the conclusion of a monitoring session, thus assuring single use of the appliance.

We claim:

1. An appliance for acquiring electroencephalograph signal from a patient comprising:
a. an insulating dielectric layer comprising a patient segment and a connection segment, said patient segment being capable of being applied solely to the patient's forehead;
b. a plurality of electrodes capable of acquiring eletroencephalograph signals from a patient on the patient segment of the dielectric layer on the side of the dielectric layer facing the patient's skin;
c. a plurality of signal traces on the side of the dielectric layer facing the patient's skin configured so that the plural signal traces electrically connect the plural electrodes to an appliance connector across the connection segment; and
d. a conducting shield layer on the side of the dielectric layer away from the patient's skin covering both the patient segment and the connection segment and connected to the appliance connector in a configuration capable of functioning such that when the appliance is electrically connected to an external device the shield layer is electrically connected to ground.

2. The appliance of claim 1 in which the connector segment forms a pigtail.

3. The appliance of claim 1 in which the plurality of electrodes numbers no greater than six.

4. The appliance of claim 1 in which the plurality of electrodes numbers no greater than five.

5. The appliance of claim 1 in which the electrodes comprise conducting hydrogel.

6. The appliance of claim 5 in which the hydrogel forming the electrodes is embedded in an open cell foam.

7. The appliance of claim 5 in which the electrodes are generally rectangular in configuration.

8. The appliance of claim 6 in which the electrodes additionally comprise an annular adhesive ring.

9. The appliance of claim 5 in which the electrodes additionally comprise volcano tips which abrade the dermis in order to lower electrode to skin impedance.

10. The appliance of claim 8 in which the adhesive pad is configured with an acute angle on the side of the adhesive pad adjacent to the patient's skin and the open cell foam comprises an acute angle on the side away from the patient's skin such that the bevel inhibits the open cell foam from being pulled out of the annular adhesive ring.

11. The appliance of claim 10 in which the configuration of the angles on the annular adhesive pad and the open cell foam is such that a small air space is formed therebetween, such that when the appliance is pressed against the patient's skin the air space receives hydrogel so as to inhibit expression of hydrogel into the space between the patient's skin and the annular adhesive pad.

12. An electrode for acquiring electrical signals representative of bodily functions from a patient's skin, said electrode comprising
a. an open cell foam pad impregnated with electrically conducting hydrogel;
b. an annular adhesive ring surrounding the open cell foam pad and having on a side which will be in contact with a patient an adhesive substance capable of temporarily adhering the annular ring to the patient's skin;
c. volcano tips in the space inside the annular adhesive ring which abrade the patient's skin so as to provide improved electrode to skin impedance.

13. The electrode of claim 12 additionally comprising angular portions on the inside of the annular adhesive ring and the outside of the open cell foam pad configured to impede the open cell foam pad from pulling out of the inside of the annular adhesive ring.

14. The electrode of claim 13 wherein the angular portions of the annular adhesive ring and the open cell foam pad are configured to leave a small air space between the two, whereby the hydrogel is impeded from extruding into the space between the adhesive substance and the patient's skin as the appliance is situated on the patient's forehead.

15. The appliance of claim 1 additionally comprising an element containing a unique identification code in electronic form and an additional signal trace capable of transmitting said unique identification code to external devices.

16. The system of claim 6, wherein the module further comprises a processor for processing physiological signals if said module recognizes said hardwired unique identification code as a supported code.
